(19) United States
(12) Patent Application Publication
(10) Pub. No.: US 2024/0299358 A1
(43) Pub. Date: Sep. 12, 2024

(54) COMPOSITIONS AND METHODS FOR DIAGNOSIS AND TREATMENT OF AUTOIMMUNE DISEASES

(71) Applicant: DUKE UNIVERSITY, Durham, NC (US)

(72) Inventors: Scott Scarneo, Durham, NC (US); Timothy Haystead, Durham, NC (US)

(21) Appl. No.: 18/260,503

(22) PCT Filed: Jan. 5, 2022
(86) PCT No.: PCT/US22/11258

§ 371 Date: Jul. 6, 2023

Related U.S. Application Data
(60) Provisional application No. 63/134,410, filed on Jan. 6, 2021.

(51) Int. Cl. A61K 31/416; A61K 47/20; A61P 37/06

(52) U.S. Cl. CPC A61K 31/416; A61K 47/20; A61P 37/06

(57) ABSTRACT

The present disclosure describes, in part, methods for diagnosing and/or treating autoimmune diseases using a compound capable of inhibiting eHsp90.

### CROSS REFERENCE TO RELATED APPLICATIONS

This PCT application claims benefit of priority to U.S. Provisional Patent Application No. 63/134,410, filed Jan. 6, 2021, the contents of which is hereby incorporated by reference in its entirety.

### FIELD

The present disclosure generally relates to medical treatment. In particular, the present disclosure relates to methods of diagnosing and treating autoimmune diseases.

### BACKGROUND

T cells play an integral role in the host adaptive immune response, choreographing immune surveillance and the host response to infection (Medzhitov & Janeway, Curr Opin Immunol 9, 4-9 (1997)). Following T cell receptor (TCR) stimulation, T cells undergo rapid replication and activation, and respond to their target antigens by secreting immunoregulatory cytokines and chemokines or through direct cell killing (Janeway, Microbes Infect 3, 1167-1171 (2001)). These rapid cellular changes are governed by TCR mediated protein kinase signal transduction, which increases the expression of specific CD markers essential to the T cell response (Gaud et al., Nat Rev Immunol 18, 485-497 (2018): Stepanek et al. Cell 159, 333-345 (2014)). Heat shock protein 90 (Hsp90) and some of its co-chaperones are upregulated during T cell activation and have been shown to play an important role in regulating many protein kinases essential for T cell activation (Schnaider et al., Life Sci 63, 949-954 (1998): Schnaider et al., Cell Stress Chaperones 5, 52-61 (2000): Delgoffe et al., Mol Immunol 46, 2694-2698 (2009)). Hsp90 inhibition has also been shown to block T cell function in vivo, as seen in mouse models of allograft rejection and in rheumatoid arthritis (Rice et al. Arthritis Rheum 58, 3765-3775 (2008)). Thus, Hsp90's role in mediating T cell proliferation is essential to T-cell mediated immune surveillance.

A membrane/ectopic form of Heat shock protein 90 (eHsp90) was recently characterized that appeared to be only expressed on the surface of rapidly proliferating tumor cells exhibiting a metastatic phenotype (Barrott et al. Chem Biol 20, 1187-1197 (2013): Tsutsumi & Neckers, Cancer Sci 98, 1536-1539 (2007): Crowe et al., ACS Chem Biol 12, 1047-1055 (2017): Wong & Jay Adv Cancer Res 129, 141-163 (2016)). This phenomenon was originally characterized by Neckers and colleagues, showing that antibodies to Hsp90 block cell migration in tumor lines expressing the surface form of the protein (Tsutsumi et al., Oncogene 27, 2478-2487 (2008)). Subsequently, using cell impermeable fluor-tethered inhibitors of Hsp90, confocal microscopy was utilized to study the trafficking of eHsp90 on the surface of various aggressive tumor cell lines (Barrott et al. Chem Biol 20, 1187-1197 (2013): Crowe et al., ACS Chem Biol 12, 1047-1055 (2017)). These studies revealed that the protein was actively trafficked to the plasma membrane (PM) under oncogene control and upon binding of a tethered Hsp90 inhibitor, formed aggregates (90 bodies) that were actively reinternalized. In vivo studies with tethered Hsp90 inhibitors in mice bearing breast tumors showed expression of eHsp90 was indeed exclusively associated with malignant tumor cells.

### SUMMARY

The present disclosure is based, in part, on the discovery that activated CD⁶⁹⁺ and/or CD²⁵⁺ T cells express high levels of eHsp90 at the plasma membrane (PM) in response to T cell activation by anti-CD3/CD28 antibody challenge as well as cytokine stimulation, suggesting that trafficking of eHsp90 is a regulated process in T cells and not a phenomenon of cellular stress such as occurs in oncogenic transformation. Expression of eHsp90 in T cell populations was followed both in vitro and in vivo with HS-131, a Cy5 carrying tethered inhibitor of Hsp90. Furthermore, systemic evaluation of eHsp90 expression in murine models of autoimmune disease shows that tissues with robust expression of eHsp90 also show histopathological evidence of active disease. The results show that targeting of eHsp90 provides a selective diagnostic means to evaluate active disease sites in autoimmunity and suggest that tethered inhibitors of Hsp90 could be developed as a means to achieve precision immunosuppression in vivo.

Accordingly, one aspect of the present disclosure provides a method of preventing or treating an autoimmune disease in a subject. This method may comprise administering to the subject a compound capable of inhibiting eHsp90.

Another aspect of the present disclosure provides a method of diagnosing an autoimmune disease in a subject. This method may comprise obtaining from the subject a biological sample, and contacting the biological sample with a compound capable of inhibiting eHsp90. In this method, detection of elevated T cell activation in the biological sample is indicative of presence of an autoimmune disease in the subject.

Another aspect of the present disclosure provides a method of delaying onset and progression of an autoimmune disease in a subject. This method may comprise administering to the subject a compound capable of inhibiting eHsp90.

Still another aspect of the present disclosure provides a method of managing a transplant rejection in a subject. This method may comprise administering to the subject a compound capable of inhibiting eHsp90 thereby tracking T cell activation.

Yet another aspect of the present disclosure provides a method of managing an infection in a subject. This method may comprise administering to the subject a compound capable of inhibiting eHsp90 thereby tracking T cell activation.

For any of the methods described above and herein, one or more additional autoimmune therapies may be co-administered to the subject.

In some embodiments, the compound used for any of the methods described above and herein may have the following general formula (I):

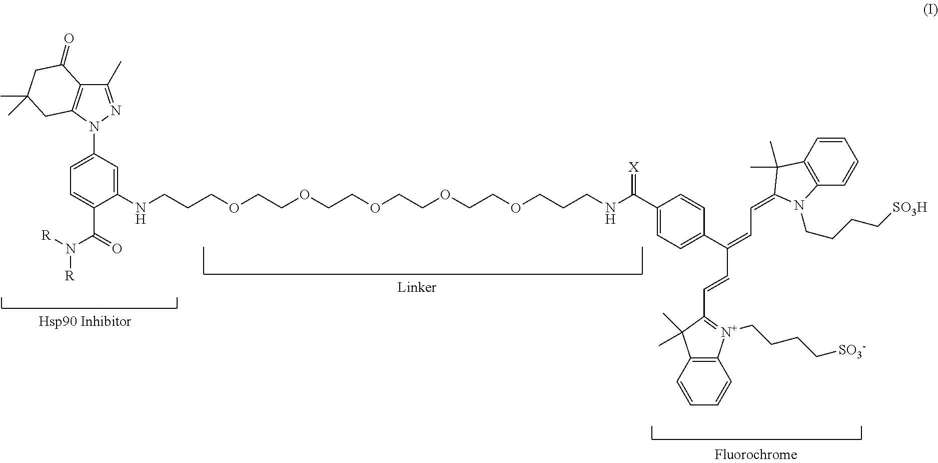

wherein R may be H or C₁₋₈-alkyl; and X may be X is H2, O, or S.

Suitable examples of the compound include, but are not limited to, HS-131 and HS-132. For HS-131, R is H, and X is H2 in above formula (I). For HS-132, R is H, and X is O in above formula (I).

In some embodiments, the autoimmune disease may be a disease that confers maladaptive T cell activation. By way of non-limiting example, the autoimmune disease may be rheumatoid arthritis (RA), osteoarthritis (OA), systemic scleroderma, Chron's disease, systemic lupus erythematosus, inflammatory bowel disease (IBD), Type I diabetes, chronic Lyme disease, Addison disease, celiac disease, dermatomyositis, Graves disease, Hashimoto thyroiditis, multiple sclerosis, Myasthenia gravis, Pernicious anemia, reactive arthritis, or Sjogren syndrome.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing aspects and other features of the invention are explained in the following description, taken in connection with the accompanying drawings, wherein:

FIGS. 1A-1C illustrate Hsp90 upregulation following T cell activation. FIG. 1A shows mRNA expression of co-chaperones and mitochondrial homologs of Hsp90 72 hours following T cell activation. FIG. 1B shows that T cell isolated from PBMC's were activated with anti-CD28 and anti-CD3 antibodies for 0, 72 or 96 hours with or without additional IL-2 stimulation and Hsp90, co-chaperones and mitochondrial homologs were probed via western blot for relative protein expression. FIG. 1C shows Hsp90 isolated from T cells 24 hours post activation and purified on a Hsp90 specific resin analyzed by MALDI mass spectrometry, for alpha and beta Hsp90 isoforms. Data represents 6 biological replicates. The data was analyzed with paired t-test. *** p<0.001.

FIGS. 2A-2E illustrate that T cell activation induces eHsp90 expression. FIG. 2A shows the chemical structure of HS-131, and HS-132 active molecule, the inactive analogue HS-198 (negative control). FIG. 2B shows that Naïve T cells isolated from healthy human peripheral blood mononuclear cells (PBMC's) were activated with anti-CD28 and anti-CD3 antibodies, or resting (unstimulated) for 96 hours and treated with HS-132 at the indicated concentrations. Data represented as mean ±SD, 6 biological replicates. FIG. 2C shows internalization of probe by T cells at physiological (37° C.) and non-physiological (4° C.) temperatures at varying concentrations of HS-132. Data represents mean ±SD, 4 biological replicates. FIG. 2D shows that T cells isolated from stimulated human PBMC's were treated with 10 μM HS-131 (active molecule) and HS-198 (inactive control) with or without a 10-fold excess of ganetespib. Data represents mean ±SD, 3 biological replicates. FIG. 2E shows confocal analysis of HS-132 uptake in stimulated T cells, which was blocked with treatment of the non-fluorochrome tethered Hsp90 inhibitor HS-10.

FIG. 3A shows temporal upregulation of eHsp90 compared to total Hsp90 levels in T cells stimulated with anti-CD3/CD28 antibodies over a 96-hour period. The data were analyzed by 2-way ANOVA with Sidak's multiple comparison test, * p<0.05, ** p<0.01. *** p<0.001. Experiments were performed with 6 biological replicates. FIG. 3B shows flow cytometric analysis of CD3, CD25, and CD69 T-cell populations relative eHsp90 expression compared to baseline. Experiments were performed with 3 biological replicates for each timepoint. FIG. 3C shows upregulation of total Hsp90 in activated T cell populations. Experiments were performed with 3 biological replicates for each timepoint. FIG. 3D shows mean (+SD) CD25 expression on T cells 24-96 hours post CD3/28 stimulation. Temporal upregulation of eHsp90 (green line) post T cell stimulation. CD25 expression: 6-8 biological replicates per time point. eHsp90 expression 6 biological replicates for each timepoint. FIG. 3E shows that eHsp90 and total Hsp90 expression in CD3+ T cells isolated from PBMC's were stimulated with IL-2 (60 IU/mL) for 24 hours. Data represent mean ±SD. The data were analyzed by 2-way ANOVA with Sidaks multiple comparison test. Experiments were performed once with 3 biological replicates. FIG. 3F shows cytokine expression 24 hours post anti-CD3/28 T cell stimulation treated with or without HS-131 (1 μM). Data represent mean ±SEM. The data were analyzed by 2-way ANOVA with Sidaks multiple comparison test *** p<0).001. #significantly different than activated-vehicle treated. Experiment was performed once with 4 biological replicates.

FIGS. 4A-4F illustrate effects of three monoclonal Hsp90 targeted antibodies (771, 902, AC88). Hsp90 antibodies were treated at varying concentrations in resting T cells (FIGS. 4A-4C) and activated T cells (anti-CD3/28 activation, FIGS. 4D-4F). % viability (FIGS. 4A and 4D), % CD25 (FIGS. 4B and 4E) and total cell count recorded (FIGS. 4C and 4F) are shown. N=3-6 biological replicates/group.

FIG. 5A is a schematic of CIA mouse model of RA experimental design, wherein mice were inoculated with CIA on day 0) and 21. At peak of disease onset (Day 32) animals received I.V. HS-131 or HS-198 (inactive control). 6-hours post treatment mice were whole body cryopreserved. FIG. 5B shows flow cytometric analysis of eHsp90 expression on CD4 and CD8 T Cells isolated from the serum of HS-131 and HS-198 treated mice 6 hours post tail vein injection. Gated regions indicate CD4 and CD8 T-Cell populations isolated from individual mice. eHsp90 expression in CD4 and CD8 T cells 6 hours post HS-131 or HS-198 treatment. N=3+SD. Experiments represent 3 biological replicates. FIG. 5C shows brightfield and fluorescent histology of hindleg, knee, hip and paw of CIA mice 6 hours post tail vein injection of HS-131. FIG. 5D shows whole body reconstruction of individual HS-131 treated CIA mice 6 hours post tail vein injection. FIG. 5E shows brightfield and fluorescent cross section of CIA mouse.

FIG. 6A illustrates eHsp90 expression in a naïve disease-free mouse injected with HS-131 (10 nmoles, tail vein) cryo-imaged 6 hours post injection. Comparison of knee joint of naïve mouse (FIG. 6B) and CIA arthritic mouse (FIG. 6C) eHsp90 expression are shown.

FIG. 7A shows eHsp90 expression in an inflammatory bowel disease (IBD) model of auto immune disease. The T cell transfer model of IBD was utilized to evaluate the imaging potential of HS-131 for other autoimmune diseases. Naïve T cells isolated from female donor mice were transferred into SCID mice via IP injection. 3-5 weeks post transfer mice develop inflammation, an extended colon and epithelial hyperplasia. 24 hours post HS-131 tail vein injection mice were cryopreserved and imaged as previously mentioned. FIG. 7B shows eHsp90 expression in a CIA RA mouse 24 hours post HS-131 injection. Experiments represent one mouse.

FIG. 8 illustrates flow cytometric gating scheme for T cell analysis in CIA mice receiving HS-131 or HS-198.

FIGS. 9A-9E illustrate therapeutic potential of eHsp90 targeted therapies for the treatment of auto immune diseases. FIG. 9A shows animals developing disease, identified by inflammation within at least one paw. FIG. 9B shows mean arthritic clinical score of HS-131 30 mg/kg IP, QD and vehicle IP, QD treated mice throughout the study duration N=12/group+SEM. FIG. 9C shows area under the curve of HS-131 treated mice compared to vehicle N=12/group+SEM. FIG. 9D are representative photomicrographs of forepaw, knee and ankle from a disease control (Naïve), diseased vehicle treated and HS-131 treated animals. “W” identifies wrist, arrows identifies representative affected joints, and “S” identifies inflammation. FIG. 9E shows HS-131 reduced pannus, inflammation, cartilage damage, bone resorption and periosteal bone histological manifestations of CIA 36 days post disease onset N=12/group+SEM. Experiment was performed once with 12 biological replicates per group.

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including.” “comprising,” or “having.” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition. As used herein, the terms “prevent,” “preventing.” “prevention,” “prophylactic treatment” and the like refer to reducing the probability of developing a disease, disorder or condition in a subject, who does not have, but is at risk of or susceptible to developing a disease, disorder or condition. The term “effective amount” or “therapeutically effective amount” refers to an amount sufficient to effect beneficial or desirable biological and/or clinical results. In some embodiments, the disease, disorder or condition comprises an autoimmune disease/disorder.

As used herein, the terms “autoimmune disease” and “autoimmune disorder” are used interchangeably and refer to any disease, disorder or condition characterized by the body's immune system attacking and destroying healthy body tissue by mistake. Such diseases, disorders and/or conditions may result in the destruction of body tissue, abnormal growth of an organ, and/or changes in organ function and may affect one or more organ or tissue types, such as blood vessels, connective tissue, endocrine glands (e.g. thyroid or pancreas), joints, muscles, red blood cells, skin and the like. A person may have more than one autoimmune disease, disorder and/or condition at the same time. Examples of autoimmune diseases, disorders and/or conditions include, but are not limited to, Addison disease, celiac disease-sprue, dermatomyositis, Graves disease, Hashimoto thyroiditis, Multiple Sclerosis, Myasthenia gravis, Pernicious anemia, reactive arthritis, rheumatoid arthritis, osteoarthritis, systemic scleroderma, Chron's disease, Sjogren syndrome, systemic lupus erythematosus, Type I diabetes, and the like.

As used herein, the term “administering” an agent, such as a therapeutic entity to an animal or cell, is intended to refer to dispensing, delivering or applying the substance to the intended target. In terms of the therapeutic agent, the term “administering” is intended to refer to contacting or dispensing, delivering or applying the therapeutic agent to a subject by any suitable route for delivery of the therapeutic agent to the desired location in the animal, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route.

As used herein, the term “biomarker” refers to a naturally occurring biological molecule present in a subject at varying concentrations useful in predicting the risk or incidence of a disease or a condition, such as an autoimmune disease, disorder and/or condition. For example, the biomarker can be a protein present in higher or lower amounts in a subject at risk for metastatic pancreatic cancer. The biomarker can include nucleic acids, ribonucleic acids, or a polypeptide used as an indicator or marker for metastatic pancreatic cancer in the subject. In some embodiments, the biomarker is a protein. In certain embodiments, the biomarker comprises eHsp90.

The term “biological sample” as used herein includes, but is not limited to, a sample containing tissues, cells, and/or biological fluids isolated from a subject. Examples of biological samples include, but are not limited to, tissues, cells, biopsies, blood, lymph, serum, plasma, urine, saliva, mucus and tears. In one embodiment, the biological sample comprises blood. A biological sample may be obtained directly from a subject (e.g., by blood or tissue sampling) or from a third party (e.g., received from an intermediary, such as a healthcare provider or lab technician).

“Contacting” as used herein, e.g., as in “contacting a sample” refers to contacting a sample directly or indirectly in vitro, ex vivo, or in vivo (i.e. within a subject as defined herein). Contacting a sample may include addition of a compound to a sample (e.g., a labeled eHsp90 inhibitor as provided herein), or administration to a subject. Contacting encompasses administration to a solution, cell, tissue, mammal, subject, patient, or human. Further, contacting a cell includes adding an agent to a cell culture.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The methods and compositions disclosed herein can be used on a sample either in vitro (for example, on isolated cells or tissues) or in vivo in a subject (i.e. living organism, such as a patient). In some embodiments, the subject comprises a human.

Unless otherwise defined, all technical terms used herein have the same meaning as commonly understood by one of ordinary skill in the art to which this disclosure belongs.

### A. Compounds

The present disclosure is based, in part, on the discovery that activated CD69+/CD25+ T cells express high levels of eHsp90 at the plasma membrane (PM) in response to anti-CD3/CD28 antibody challenge, and that targeting eHsp90 with eHsp90 inhibitory compounds provides a selective diagnostic means to evaluate active disease sites in autoimmunity and that tethered inhibitors of Hsp90 can be used as a means to achieve precision immunosuppression in vivo.

Accordingly, used in the present disclosure is a composition comprising, consisting of, or consisting essentially of a compound capable of inhibiting eHsp90. Suitable examples of eHsp90 inhibitory compounds include, but are not limited to, those disclosed in U.S. Pat. No. 10,442,806, the content of which is hereby incorporated by reference in their entirety. The eHsp90 inhibitory compound may further comprise a biological label attached to the compound by a linker. Any suitable biological label can be used that allows for the visualization and/or tracking of a compound in vitro, ex vivo, and/or in vivo. Suitable examples of a biological label include, but are not limited to, a fluorophore.

In some embodiments, the compound capable of inhibiting eHsp90 may have the general formula (I):

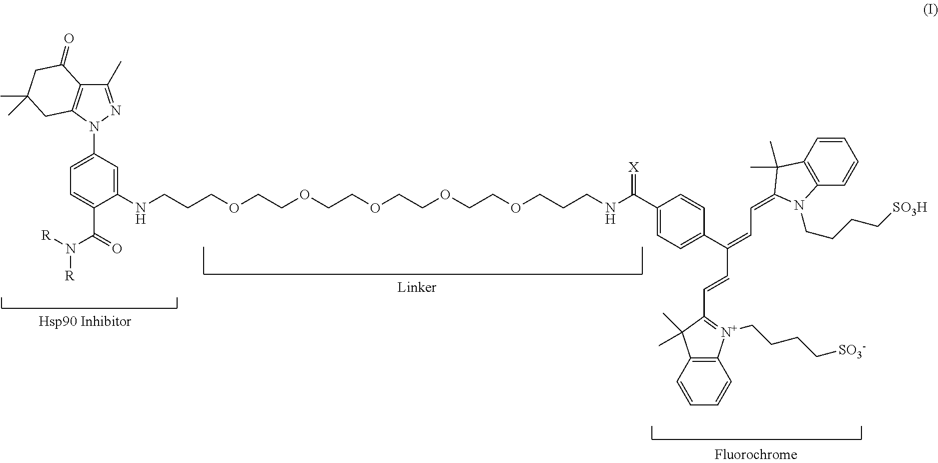

wherein R may be H or C₁₋₈-alkyl; and X may be X is H₂, O, or S. The compound may be a pharmaceutically acceptable salt, solvate, hydrate, prodrug, or derivative thereof.

Suitable examples of the compound include, but are not limited to, HS-131 and HS-132. For HS-131, R is H, and X is H₂ in above formula (I). For HS-132, R is H, and X is O in above formula (I).

### B. Pharmaceutical Compositions

Also used in the present disclosure are compositions comprising one or more of compounds as described herein and an appropriate carrier, excipient or diluent. The exact nature of the carrier, excipient or diluent will depend upon the desired use for the composition, and may range from being suitable or acceptable for veterinary uses to being suitable or acceptable for human use. The composition may optionally include one or more additional compounds.

When used to treat or prevent a disease, such as an autoimmune disease, disorder and/or condition, the compounds described herein may be administered singly, as mixtures of one or more compounds or in mixture or combination with other agents (e.g., therapeutic agents) useful for treating such diseases and/or the symptoms associated with such diseases. Such agents may include, but are not limited to, antibiotics, NSAIDS, anti-inflammatory compounds, immune-suppressing drugs (e.g., corticosteroids, biologics, monoclonal antibodies, Janus Kinase inhibitors, Calcineurin inhibitors, mTOR inhibitors, IMDH inhibitors, etc.), and surgical intervention, to name a few. The compounds may be administered in the form of compounds per se, (e.g., as an eHsp90 inhibitor and/or an eHsp90 inhibitor further comprising a biological label attached to the inhibitor via a linker), or as pharmaceutical compositions comprising said compounds.

Pharmaceutical compositions comprising the compound(s) may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients or auxiliaries which facilitate processing of the compounds into preparations which can be used pharmaceutically.

The compounds may be formulated in the pharmaceutical composition per se, or in the form of a hydrate, solvate, N-oxide or pharmaceutically acceptable salt, as previously described. Typically, such salts are more soluble in aqueous solutions than the corresponding free acids and bases, but salts having lower solubility than the corresponding free acids and bases may also be formed.

Pharmaceutical compositions may take a form suitable for virtually any mode of administration, including, for example, topical, ocular, oral, buccal, systemic, nasal, injection, transdermal, rectal, vaginal, etc., or a form suitable for administration by inhalation or insufflation.

For topical administration, the compound(s) may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives. Alternatively, the injectable formulation may be provided in powder form for reconstitution with a suitable vehicle, including but not limited to sterile pyrogen free water, buffer, dextrose solution, etc., before use. To this end, the active compound(s) may be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients such as binding agents (e.g., pregelatinised maize starch, polyvinylpyrrolidone or hydroxypropyl methylcellulose): fillers (e.g., lactose, microcrystalline cellulose or calcium hydrogen phosphate): lubricants (e.g., magnesium stearate, talc or silica): disintegrants (e.g., potato starch or sodium starch glycolate): or wetting agents (e.g., sodium lauryl sulfate). The tablets may be coated by methods well known in the art with, for example, sugars, films or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats): emulsifying agents (e.g., lecithin or acacia): non-aqueous vehicles (e.g., almond oil, oily esters, ethyl alcohol, cremophore™ or fractionated vegetable oils); and preservatives (e.g., methyl or propyl-p-hydroxy benzoates or sorbic acid). The preparations may also contain buffer salts, preservatives, flavoring, coloring and sweetening agents as appropriate.

Preparations for oral administration may be suitably formulated to give controlled release of the compound, as is well known. For buccal administration, the compositions may take the form of tablets or lozenges formulated in conventional manner. For rectal and vaginal routes of administration, the compound(s) may be formulated as solutions (for retention enemas) suppositories or ointments containing conventional suppository bases such as cocoa butter or other glycerides.

For nasal administration or administration by inhalation or insufflation, the compound(s) can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

For ocular administration, the compound(s) may be formulated as a solution, emulsion, suspension, etc. suitable for administration to the eye. A variety of vehicles suitable for administering compounds to the eye are known in the art.

For prolonged delivery, the compound(s) can be formulated as a depot preparation for administration by implantation or intramuscular injection. The compound(s) may be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the compound(s) for percutaneous absorption may be used. To this end, permeation enhancers may be used to facilitate transdermal penetration of the compound(s).

Alternatively, other pharmaceutical delivery systems may be employed. Liposomes and emulsions are well-known examples of delivery vehicles that may be used to deliver compound(s). Certain organic solvents such as dimethyl sulfoxide (DMSO) may also be employed, although usually at the cost of greater toxicity.

The pharmaceutical compositions may, if desired, be presented in a pack or dispenser device which may contain one or more unit-dosage forms containing the compound(s). The pack may, for example, comprise metal or plastic foil, such as a blister pack. The pack or dispenser device may be accompanied by instructions for administration.

The compound(s) described herein, or compositions thereof, will generally be used in an amount effective to achieve the intended result, for example in an amount effective to treat or prevent the particular disease being treated. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized.

The amount of compound(s) administered will depend upon a variety of factors, including, for example, the particular indication being treated, the mode of administration, whether the desired benefit is prophylactic or therapeutic, the severity of the indication being treated and the age and weight of the patient, the bioavailability of the particular compound(s) the conversation rate and efficiency into active drug compound under the selected route of administration, etc.

Determination of an effective dosage of compound(s) for a particular use and mode of administration is well within the capabilities of those skilled in the art. Effective dosages may be estimated initially from in vitro activity and metabolism assays. For example, an initial dosage of compound for use in animals may be formulated to achieve a circulating blood or serum concentration of the metabolite active compound that is at or above an IC₅₀ of the particular compound as measured in as in vitro assay. Calculating dosages to achieve such circulating blood or serum concentrations taking into account the bioavailability of the particular compound via the desired route of administration is well within the capabilities of skilled artisans. Initial dosages of compound can also be estimated from in vivo data, such as animal models. Animal models useful for testing the efficacy of the active metabolites to treat or prevent the various diseases described above are well-known in the art. Animal models suitable for testing the bioavailability and/or metabolism of compounds into active metabolites are also well-known. Ordinarily skilled artisans can routinely adapt such information to determine dosages of particular compounds suitable for human administration.

Dosage amounts will typically be in the range of from about 0.0001 mg/kg/day, 0.001 mg/kg/day or 0.01 mg/kg/day to about 100 mg/kg/day, but may be higher or lower, depending upon, among other factors, the activity of the active compound, the bioavailability of the compound, its metabolism kinetics and other pharmacokinetic properties, the mode of administration and various other factors, discussed above. Dosage amount and interval may be adjusted individually to provide plasma levels of the compound(s) and/or active metabolite compound(s) which are sufficient to maintain therapeutic or prophylactic effect. For example, the compounds may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective dosages without undue experimentation.

### C. Methods

The compounds/compositions described above and herein may be used in both diagnostic and treatment methods. Accordingly, one aspect of the present disclosure provides a method of preventing or treating an autoimmune disease in a subject. This method may comprise administering to the subject a compound capable of inhibiting eHsp90 as described above and herein.

Another aspect of the present disclosure provides a method of diagnosing an autoimmune disease in a subject. This method may comprise obtaining from the subject a biological sample, and contacting the biological sample with a compound capable of inhibiting eHsp90. In this method, detection of elevated T cell activation in the biological sample is indicative of presence of an autoimmune disease in the subject.

In some embodiments, the determining of the presence of a condition comprises detecting a signal. A signal may be detected by any suitable means appropriate for the compound. For example, a signal may be detected using a fluorometer or a fluorescence plate reader, or by using fluorescence techniques such as fluorescence microscopy, fluorescence resonance energy transfer, flow cytometry and fluorescence-activated cell sorting. In some embodiments, a signal may be detected using scintillation counting or radioimaging techniques. In some embodiments, a signal may be detected using positron emission tomography. A signal may be quantitated, for example, by comparing the quantity of the signal to that of a reference sample.

A reference sample may be a sample from a healthy subject, i.e. a subject having no clinical signs or symptoms of cancer. Suitably, the healthy subject may be clinically evaluated for otherwise undetected signs or symptoms of an autoimmune disease, disorder and/or condition, which evaluation may include routine physical examination and/or laboratory testing.

Another aspect of the present disclosure provides a method of delaying onset and progression of an autoimmune disease in a subject. This method may comprise administering to the subject a compound capable of inhibiting eHsp90.

Still another aspect of the present disclosure provides a method of managing a transplant rejection in a subject. This method may comprise administering to the subject a compound capable of inhibiting eHsp90 thereby tracking T cell activation.

Yet another aspect of the present disclosure provides a method of managing an infection in a subject. This method may comprise administering to the subject a compound capable of inhibiting eHsp90 thereby tracking T cell activation.

In some embodiments, any of the methods described above and herein may further comprise administering to the subject one or more additional autoimmune therapies.

### D. Kits

In another aspect, the disclosure provides a kit, which may be used for detecting Hsp90 in a sample, for detecting an autoimmune disease, disorder and/or condition in a sample, or for treating an autoimmune disease, disorder and/or condition in a subject.

In one embodiment, a kit will include a compound/composition as described herein. A kit may also include instructions for use of the compound/composition. Instructions included in kits can be affixed to packaging material or can be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD, DVD), and the like. As used herein, the term “instructions” can include the address of an internet site that provides the instructions.

In one embodiment, the disclosure provides a kit for detecting eHsp90 in a sample. The kit comprises at least one compound as provided herein and instructions for assaying the test sample for eHsp90. For example, the kit can comprise instructions for assaying the test sample for eHsp90 by fluorescence detection. The kit may further comprise a calibrator or control, e.g., purified, and optionally lyophilized, (e.g., eHsp90), and/or at least one container (e.g., tube, microtiter plates or strips) for conducting the assay, and/or a buffer, such as an assay buffer or a wash buffer, either one of which can be provided as a concentrated solution. Preferably, the kit comprises all components, i.e., reagents, standards, buffers, diluents, etc., which are necessary to perform the assay. The instructions also can include instructions for generating a standard curve or a reference standard for purposes of quantifying eHsp90.

The kit can also optionally include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), also can be included in the kit. The kit can additionally include one or more other controls. One or more of the components of the kit can be lyophilized, in which case the kit can further comprise reagents suitable for the reconstitution of the lyophilized components.

The various components of the kit optionally are provided in suitable containers as necessary, e.g., a microtiter plate. The kit can further include containers for holding or storing a sample (e.g., a container or cartridge for a blood sample). Where appropriate, the kit optionally also can contain reaction vessels, mixing vessels, and other components that facilitate the preparation of reagents or the test sample. The kit can also include one or more instrument for assisting with obtaining a test sample, such as a syringe, pipette, forceps, measured spoon, or the like.

The following Examples are provided by way of illustration and not by way of limitation.

### Materials and Methods

T-cell activation: PBMC aliquots (ZenBio SER-PBMC-F) were washed in RPMI-1640 with 10% FBS and PSG and then rested overnight in a T-75 flask. The suspended cells were then transferred to a fresh flask and activated in fresh RPMI-1640/10% FBS/PSG by 3:20,000 anti-CD3 (BioLegend 317303), 3:20,000 anti-CD28 (BioLegend 302913), and 1:10,000 (30 IU/mL) human IL-2 (CST 8907SC) and incubated for up to 72 hours. In the experiments with longer activation, samples were diluted 1:3 into fresh media containing hIL-2 at 72 hours post initial stimulation with CD3/CD28 antibodies. Rested cells were treated similarly except they did not receive activating antibody or hIL-2.

RNA-Seq Data: All RNA-Seq data was obtained on the NCBI GEO DataSets repository Accession number:GSE48978 ID:200048978.

Activation inhibition experiment-Hsp90 antibodies: ADI-SPS-771, 9D2, and AC88 (all Enzo) were cleaned with 10 kD MWCO spin filters. Cleaned antibody was added in a titration to newly activated or resting PBMCs. The cells were incubated for 72 hours and then stained for flow cytometry.

Western Blot: Samples were prepared as described in the specific experiment and then loaded on Criterion 4-15% Tris-HCl gels (BioRad 5678083 and 5678084) and separated at 200V. The proteins were transferred from the gel to a PVDF membrane (BioRad 1620177) on ice for 1 hour at 100V. After transfer, the membranes were rinsed in 1x TBST and then blocked for 30 minutes with either 5% NFDM/TBST or 5% BSA/TBST depending on the block being used for the primary antibody staining step. After blocking, the membranes were labelled with primary by overnight incubation with the manufacturer recommended concentration and blocking agent for each antibody (see, Table 1). Residual primary antibody was removed by 5-minute washes with TBST (3×) and then the appropriate secondary antibody (CST 7074 and 7076) was added at 1:10,000 in 5% NFDM/TBST for 1 hour at room temperature. Upon completion of secondary stain, residual secondary antibody was removed by 5-minute washes with TBST (3x). Clarity ECL (Biorad 1705060) reagent was used to develop film.

TABLE 1
Western Blot Antibodies

| Antibody | Target | Manufacturer | Buffer | Concentration | Secondary |
|---|---|---|---|---|---|
| PA5-27596 | Trap1 | Thermo Fisher | 5% NFDM | 1:1000 | Anti-rabbit |
| ab3280 | β-Actin | Abcam | 5% BSA | 1:5000 | Anti-mouse |
| H-114 | Hsp90α/β | Santa Cruz | 5% NFDM | 1:200 | Anti-rabbit |
| ab56721 | Aha1 | Abcam | 5% BSA | 1:1000 | Anti-rabbit |
| 5670 | Hop | Cell Signaling | 5% BSA | 1:1000 | Anti-rabbit |
| 2080 | Chip | Cell Signaling | 5% BSA | 1:1000 | Anti-rabbit |
| 11826s | FKBP4 | Cell Signaling | 5% BSA | 1:1000 | Anti-rabbit |
| 12210 | FKBP5 | Cell Signaling | 5% NFDM | 1:1000 | Anti-rabbit |
| D4I5T | Tyk2 | Cell Signaling | 5% BSA | 1:1000 | Anti-rabbit |
| 5174S | GAPDH | Cell Signaling | 5% NFDM | 1:1000 | Anti-Rabbit |

Mass Spectrometry: Following silver stain of SDS-PAGE, visible bands were excised from the gel. Gel pieces were distained using 1:1 30 mM potassium ferricyanide: 100 mM sodium thiosulfate for 10 minutes followed by alternating washes with 25 mM ammonium bicarbonate and acetonitrile. Following dehydration with acetonitrile, 25 μL of porcine trypsin (Promega)at a concentration of 4 μg/mL was added to the gel pieces. Following digestion, the supernatant was transferred to a second tube, and acetonitrile was added to the gel pieces to complete the extraction of digested peptides. This extract was added to the first supernatant and this combined solution, containing the extracted peptides was frozen and lyophilized. The peptides were resuspended in 5 μL of 100:99:1 acetonitrile: water: trifluoroacetic acid immediately prior to spotting on the MALDI target.

For MALDI analysis, the matrix solution consisted of alpha-cyano-4-hydroxycinnamic acid (Aldrich Chemical Co. Milwaukee, WI) saturating a solution of 1:1:0.01 acetonitrile: 25 mM ammonium citrate: trifuoroacetic acid. Approximately 0.15 μL of peptide solution was spotted on the MALDI target immediately followed by 0.15 μL of the matrix solution. MALDI MS and MS/MS data was then acquired using the ABSCIEX TOF/TOF. 5800 Mass Spectrometer. Resultant peptide mass fingerprint and peptide sequence data was submitted to the UniProt database using the Mascot search engine to which relevance is calculated and scores are displayed.

Flow Cytometry: The activated/rested cells were plated into a 96-well round bottom plate and pelleted at low speed. Cells were resuspended in chilled media containing titrated HS-131 or HS-198 (in 1% DMSO) and stained for 30 minutes at RT. Fluorochrome tagged inhibitors and media were washed away with a PBS wash, followed by a PBS wash containing 3% NMS. Surface antibody stains were performed in 3% NMS/PBS for 30 minutes followed by PBS washes (2x). Samples were analyzed on BD FACSCanto II flow cytometer. Permeabilized samples were prepared as previously described followed by fixation in 4% Formaldehyde/PBS overnight at 4° C. Sample analysis was performed with Flowing Software and FlowJo. Table 2 below shows the flow cytometry antibodies that were used.

TABLE 2
Flow Cytometry Antibodies

| Antibody | Target and fluor | Manufacturer | Concentration |
|---|---|---|---|
| 300434 | CD3- BV421 | BioLegend | 1:200 |
| 302616 | CD25-AF488 | BioLegend | 1:200 |
| 423113 | Viability-BV421 | BioLegend | 1:1000 |
| 423102 | Viability-BV510 | BioLegend | 1:1000 |
| 310903 | CD69-FITC | BioLegend | 1:200 |
| 302605 | CD25-PE | BioLegend | 1:200 |
| 561105 | Ly6G-AF488 | BD Biosciences | 1:500 |
| 103139 | CD45-BV605 | BioLegend | 1:800 |
| 563415 | IA/IE-BV650 | BD Biosciences | 1:1500 |
| 103121 | CD45-AF488 | BioLegend | 1:1000 |
| 300454 | CD3-AF488 | BioLegend | 1:1000 |
| 301817 | CD14-AF488 | BioLegend | 1:1000 |
| 400625 | Rat IgG2b Isotype | BioLegend | 1:1000 |
| | Control | | |

CIA Disease: Collagen was prepared at 4 mg/ml in 0.01 N acetic acid. Equal volumes of 4 mg/ml collagen and 5 mg/ml Freund's complete adjuvant were emulsified by hand mixing with syringes for approximately 5 min, at which point a bead of this material holds its form when placed in water. On study days 0 and 21, animals were anesthetized with isoflurane and given intradermal injections of a total of 400 μg of type II collagen in Freund's complete adjuvant at the base of the tail.

CIA Experimental Design: Mice were randomized into treatment groups by body weight on study day 18. Animals were treated from day 21-36 of the study with either 20 μL of HS-131 (30 mg/kg) PO, QD or vehicle (DMSO) PO, QD. On study day 36, the mice were euthanized for necropsy. Clinical scores were given for each of the paws (right front, left front, right rear, left rear) on study days 21-36. 0=normal, 1=one hind or fore paw joints affected or minimal diffuse erythema and swelling, 2=two hind or fore paw joints affected or moderate diffuse erythema and swelling, 3=three hind or fore paw joints affected or moderate diffuse erythema and swelling, 4=four hind or fore paw joints affected or marked diffuse erythema and swelling, 5=entire paw affected, severe diffuse erythema and severe swelling, unable to flex digits. Experimenter was blinded from the treatment group during clinical evaluation and scoring.

IBD Disease: On study day 0, Balb/C mice were terminated, and spleens obtained for CD4+CD45RBhigh cell isolation. After cells had been sorted and obtained, each animal received an IP injection of at a minimum 4×10⁵ cells (200 μl/mouse injections). On study day 49, a diseased mouse was treated IV with 20 μl of inflammation tracer HS-131 (10 nmoles). The animal was observed for 24 hours.

On study Day 50, the mouse was euthanized via CO2 inhalation, frozen in liquid nitrogen using a black cryoprotectant gel (embedding medium), and sent to BioIn Vision for analysis.

CryoViz™ Imaging: Whole mice were imaged at 10.23 μm×10.23 μm in-plane resolution using an Olympus MVX-10 microscope with a 1X objective and 0.63× magnification and 40 μm section thickness, using the Cryo Viz™ (BioInVision, Inc., Cleveland, USA). CryoViz™ is a fully automated, serial sectioning-and-imaging system which provides 3-dimensional, tiled, microscopic anatomical bright field and molecular fluorescence images over large fields-of-view such as a whole mouse. Images were acquired using a dual band FITC/TxRed fluorescence filter (Chroma, Inc., Rockingham, VT), a liquid crystal RGB filter and a low-noise monochrome camera. Raw images acquired by the Cryo Viz™ were processed to generate 3D color anatomical brightfield and molecular fluorescence volumes using the Cryo Viz™ Preprocessor software (BioIn Vision, Inc., Cleveland, Ohio). These 3D volumetric image data were then processed using the CryoViz™ 3D Visualizer software (BioInVision, Inc., Cleveland, USA) to obtain 3D reconstructed brightfield and fluorescence volume renderings and movies with 2D slice cutaway animations, showing the 2D/3D biodistribution of TxRed labeled HS-131 within the mouse volume.

Confocal Microscopy: T cells isolated from PBMC's were plated onto coverslips. The cells were stained with HS-131 and HS-198 at room temperature for 2 hours. Following staining, cells were fixed to coverslips with 4% formaldehyde/PBS. The fixative was then removed with PBS washes (3×) and Unbound stain was removed by PBS washes (2×) and then the coverslips were rinsed with ddH2O and affixed to slides with FluorSave (Millipore 345789). Imaging was performed on a Leica SP5 confocal microscope.

Immunohistological Staining: After 24-48 hours in fixative and 4-5 days in 5% formic acid for decalcification, tissues were trimmed, and processed for paraffin embedding. Paws were embedded in paraffin in the frontal plane and the knees were embedded with the patella facing down. Ankles, if left attached to the hind paw, were also embedded in the frontal plane but may be detached and sectioned in the sagittal plane for special purposes. Sections were cut and stained with toluidine blue.

### Scores for Synovitis, Pannus Formation, Degradation of Cartilage, and Bone

### Paw Score Criteria

0=Normal. 0.5=Very minimal, affects only 1 joint or minimal multifocal periarticular infiltration of inflammatory cells. 1=Minimal infiltration of inflammatory cells in synovium and periarticular tissue of affected joints. 2=Mild infiltration of inflammatory cells. When referring to paws, generally restricted to affected joints (1-3 affected). 3=Moderate infiltration with moderate edema. When referring to paws, restricted to affected joints, generally 3-4 joints and the wrist or ankle. 4=Marked infiltration affecting most areas with marked edema, 1 or 2 unaffected joints may be present. 5=Severe diffuse infiltration with severe edema affecting all joints (to some extent) and periarticular tissues.

### Knee Score Criteria

0=Normal. 0.5=Very minimal, affects only one area of the synovium or minimal multifocal periarticular infiltration of inflammatory cells. 1=Minimal infiltration of inflammatory cells in synovium and periarticular tissue of affected synovial areas. 2=Mild diffuse infiltration of inflammatory cells. 3=Moderate diffuse infiltration of inflammatory cells. 4=Marked diffuse infiltration of inflammatory cells. 5=Severe diffuse infiltration of inflammatory cells.

### Cartilage Damage Score Criteria

0=Normal. 0.5=Very minimal=Affects marginal zones only of one to several areas (knees) or joints (paws). 1=Minimal=Generally minimal to mild loss of toluidine blue staining (proteoglycan) with no obvious chondrocyte loss or collagen disruption in affected joints/areas. 2=Mild=Generally mild loss of toluidine blue staining (proteoglycan) with focal areas of chondrocyte loss and/or collagen disruption in some affected joints/areas. Paws may have one or two digit joints with near total to total loss of cartilage. 3=Moderate=Generally moderate loss of toluidine blue staining (proteoglycan) with multifocal chondrocyte loss and/or collagen disruption in affected joints/areas. Paws may have three or four joints with near total or total loss. In the knee, some matrix remains on any affected surface with areas of severe matrix loss. 4=Marked=Marked loss of toluidine blue staining (proteoglycan) with multifocal marked (depth to deep zone or tidemark) chondrocyte loss and/or collagen disruption in most joints with a few unaffected or mildly affected. In the knee, one surface with total to near total cartilage loss. 5=Severe=Severe diffuse loss of toluidine blue staining (proteoglycan) with severe (depth to tide mark) chondrocyte loss and/or collagen disruption in most or all joints.

Quantification and Statistical Analysis: Graphpad Prism 8 was used for statistical analysis of T cell activation, eHsp90 expression and viremia. For each analysis, total n and SEM are presented in the figure legend. An alpha of 0.05 was used for all statistical analysis.

### Example 1: Regulation of Hsp90 Chaperone Machinery in T cells

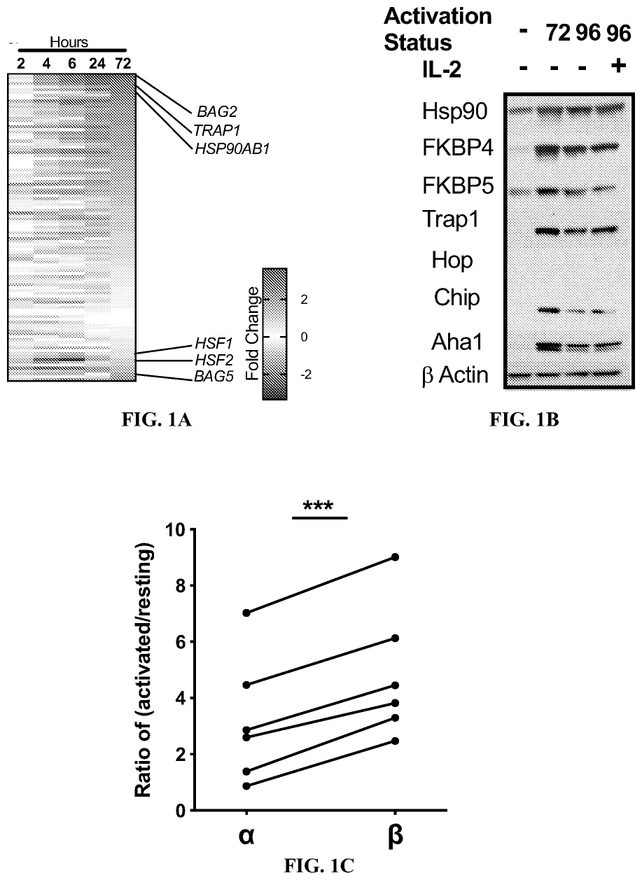

Using a list of 140 known genes that comprise the entire cellular chaperone machinery, available genome wide RNA-seq data derived from activated CD4+/CCR6+human peripheral blood mononuclear cells (PBMCs) (Wong & Jay, Adv Cancer Res 129, 141-163 (2016): Zhao et al., PLOS One 9, e78644 (2014)) were interrogated publicly. It was discovered that expression of both isoforms (a and B) of Hsp90 were highly upregulated over the time-course of activation, along with other stress induced chaperones including Hsp70, Hsp60, and Hsp40 (FIG. 1A). Significant expression changes were previously found in several Hsp90 co-chaperones including AHSAI, FNIP2, STIP1, FKBP4, PPID, and PP5 (Cortes et al., Methods Mol Biol 1709, 321-329 (2018)). Others such as FNIP1, STUB1, and FKBP5 were reduced in expression. The increased expression of known activating and regulatory proteins of Hsp90 such as FKBP4/5, Trap1, Hop, Chip, and Ahal was confirmed by western blot (FIG. 1B). Consistent with the RNA-seq data, these regulatory and co-chaperone proteins were significantly upregulated at 72 and 96 hours post stimulation compared to resting T cells. The addition of exogenous IL-2 at 72 hours post stimulation did not affect these changes in expression. Consistent with expression analysis, activated human PBMC T cell populations showed a marked induction of both Hsp90x and Hsp90ß protein by 3 and 4.5-fold respectively (p<0.001) (FIG. 1C).

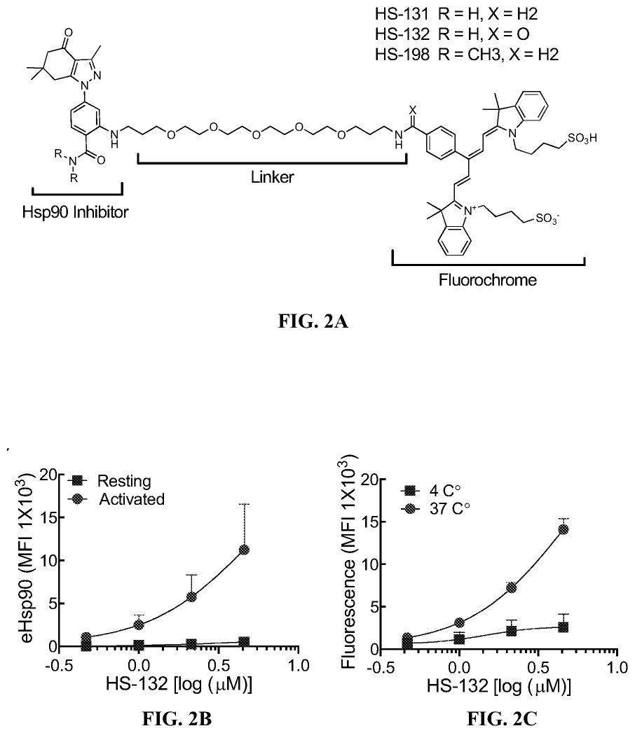

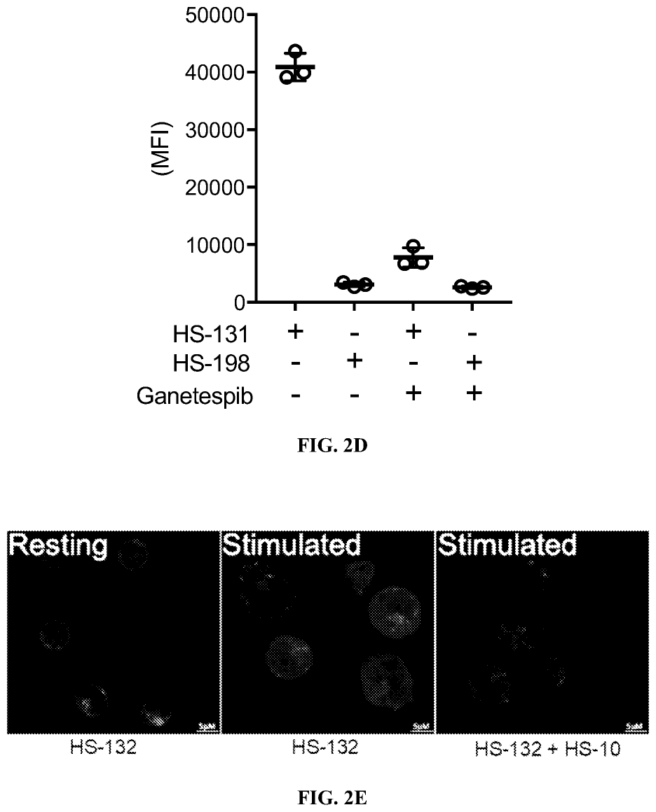

A hallmark of T cell function is to proliferate rapidly in response to antigen presentation along with appropriate cytokine and costimulatory signaling. This leads to the induction of the chaperone machinery which is essential in facilitating this process. In some respects, antigen induced T cell proliferation may be analogous to oncogene driven metastasis. Therefore, to investigate whether proliferating T cells similarly express eHsp90 during T cell activation, their ability to absorb fluor-tethered Hsp90 inhibitors following an anti-CD3/CD28 challenge was examined (see. FIGS. 2A-2E). The structures of two types of fluor-tethered Hsp90 inhibitors (HS-131 and HS-132) that were used in the present studies are shown in FIG. 2A. The probes consist of an Hsp90-binding ligand, a polyethylene-based tether (linker), and a fluorophore (Cy5 dye ex640 nm/em680 nm). Both the ligand and the fluorophore moieties can be substituted with a variety of small molecules. For example, in the present studies, three types of Cy5 probe were synthesized in which the ligand was either substituted to an N′N dimethylamide to create an inactive control molecule (HS-198) or equally potent but cold-soluble (4° C.) analog by substituting the benzylamine (HS-131) with a benzamide moiety (HS-132). Flow analysis of human PBMCs treated with or without CD3/CD28 antibodies showed dose dependent uptake of HS-132 in the activated T cell population, in contrast to resting T cells (FIG. 2B). When the study was repeated at 4° C. versus 37° C. uptake of the probe was blocked at 4° C. suggesting that uptake involves active cellular processes rather than simple diffusion (FIG. 2C). Similar temperature dependent effects were observed previously with malignant tumor cell lines (Crowe et al. ACS Chem Biol 12. 1047-1055 (2017)). The uptake of the probe appears to be eHsp90 specific, since minimal uptake was observed with the HS-198 inactive analogue (see. FIG. 2D).

Additionally, the fluor signal was effectively blocked by competition with a saturating dose of a structurally non-related ATP competitive inhibitor of Hsp90, ganetespib (Jhaveri & Modi. Onco Targets Ther 8. 1849-1858 (2015): Ying et al. Mol Cancer Ther 11. 475-484 (2012)). Therefore, activated T cells specifically internalize fluor-tethered Hsp90 inhibitors due to interactions with the ATP binding site of eHsp90 expressed at the plasma membrane. Confocal analysis of isolated activated human T cells confirms uptake of HS-132 following CD3/28 stimulation compared to resting (inactivated) T cell populations and that the binding of HS-132 was eliminated by HS-10, a non-fluorescent Hsp90 inhibitor. (see. FIG. 1E).

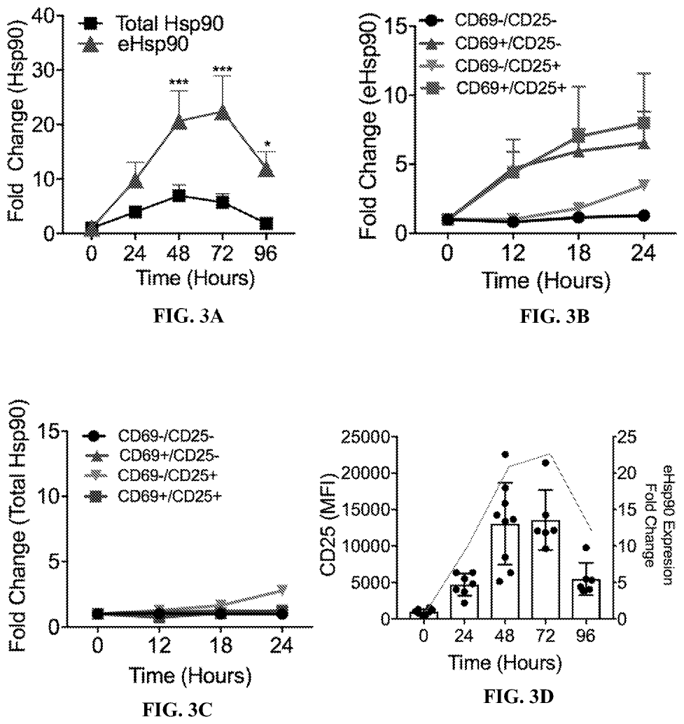

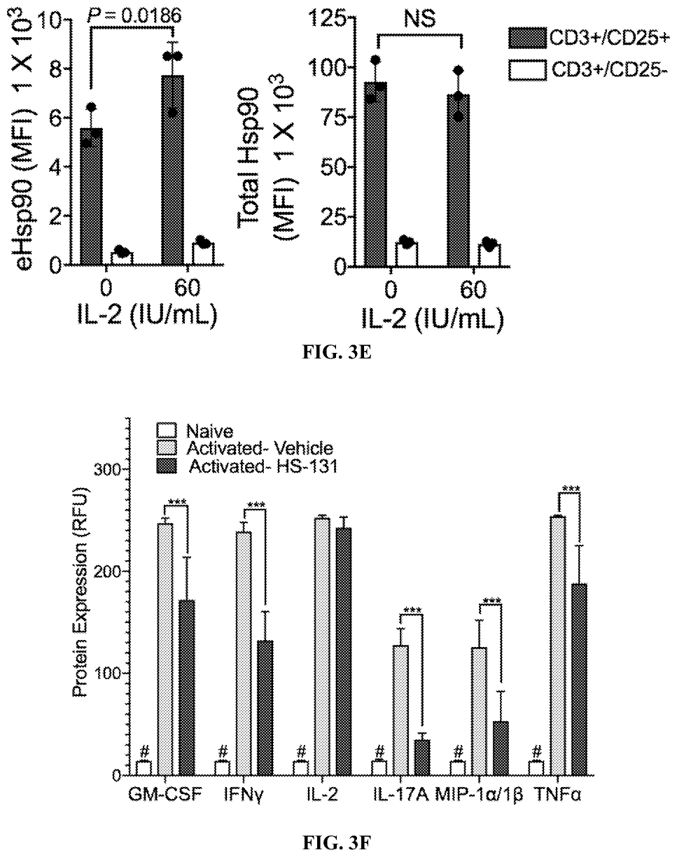

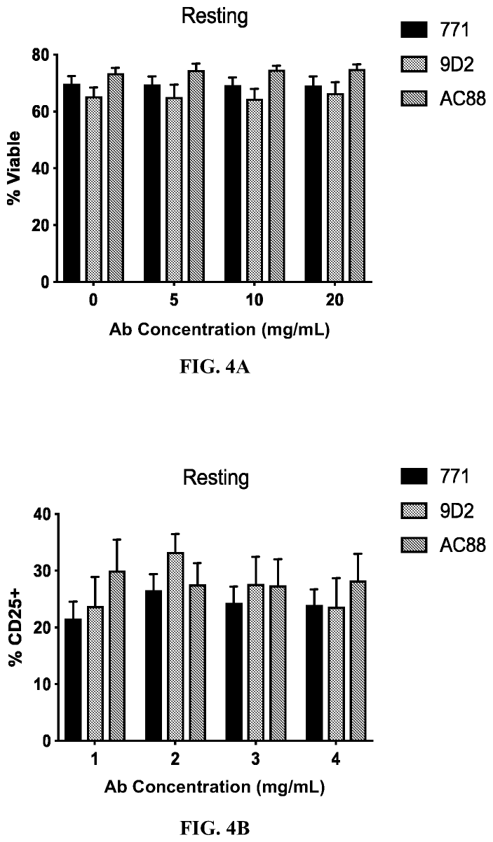

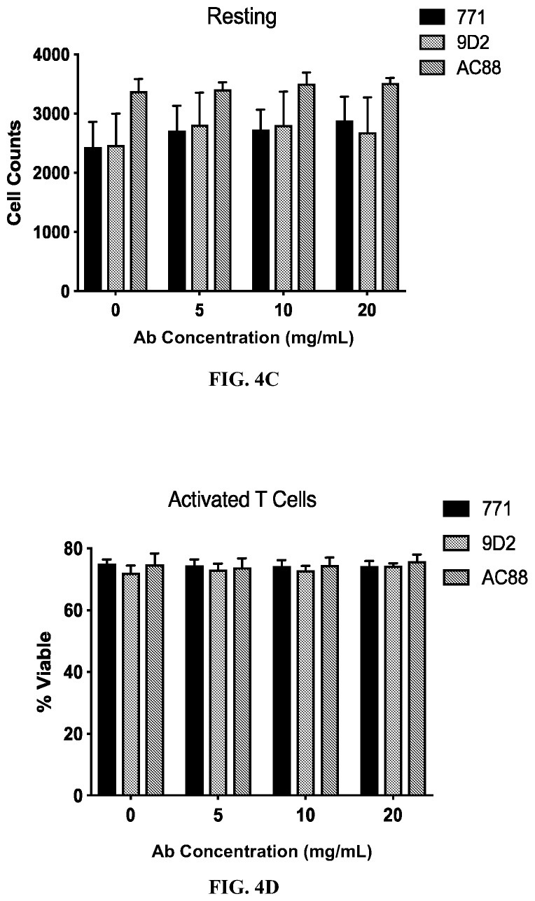

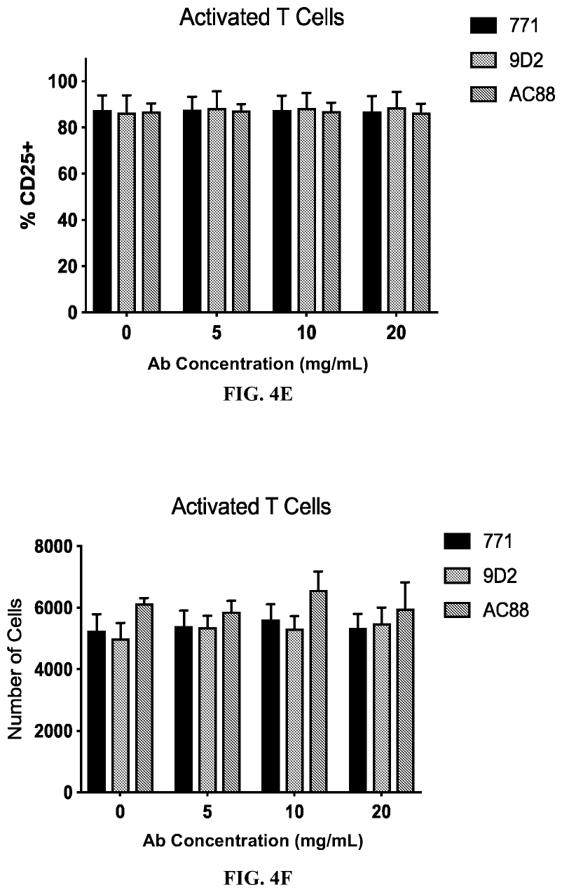

Next, the expression of eHsp90 and total Hsp90 over the course of T cell activation was tracked. Both total Hsp90 cellular content and eHsp90 expression in anti-CD3/28 stimulated human PBMCs were evaluated. Total Hsp90 expression was upregulated 5-7 fold in CD3+ T cell at 2 to 3 days post activation, followed by a reduction in Hsp90 expression. In contrast, eHsp90 expression, as determined by HS-131 binding, was upregulated 20-25 fold following activation, which is significantly more than the observed change in total Hsp90 expression (see. FIG. 3A). Specifically, anti-CD3/28 stimulation caused eHsp90 up regulation in CD69+/CD25+ and CD69+/CD25− T-cell populations with a 5-8 fold increase within the first 24 hours post activation, whereas CD69−/CD25− T cells show no increase in probe or eHsp90 levels (see. FIG. 3B). Total Hsp90 was also upregulated in the CD69−/CD25+ populations but only by approximately ˜2.5 fold (see. FIG. 3C). When eHsp90 entry was blocked with monoclonal Hsp90 antibodies, no change in viability, the percentage of CD25+ cells or even overall cell numbers was observed, indicating blocking of eHsp90 internalization does not interfere with T-cell activation (see. FIGS. 4A-4F). This data suggests that eHsp90 may function in the delivery of client proteins to the plasma membrane. One candidate client eHsp90 protein is CD25, the IL-2 receptor. This protein is highly upregulated during T-cell activation due to a positive feedback loop mediated by autocrine and exocrine IL-2 (Scarneo et al. Arthritis Res Ther 21. 292 (2019)). Here it was observed that CD25 expression peaked at 72 hours post activation, followed by a reduction in expression thereafter which correlates with reduced eHsp90 expression (see. FIG. 3D). Due to the tight correlation of CD25 and eHsp90 expression, it further tested whether exogenous IL-2 stimulation could maintain high expression of both proteins in activated T cells 72-96 hours post-stimulation. The results show that CD25 expression was significantly upregulated along with eHsp90 in a dose dependent manner in response to IL-2 (p=0.0186) (see. FIG. 3E). Total cellular Hsp90 expression was not affected by IL-2, suggesting that the trafficking of eHsp90 to the cell surface in T cells is part of a receptor mediated process rather than a reflection of changes in overall Hsp90 protein expression (see. FIG. 3E). Due to IL-2's ability to enhance eHsp90 expression in activated cells, the impacts of eHsp90 inhibition in activated T cells on inflammatory signaling were investigated. Human CD3+ T cells were stimulated with CD3/CD28 activating antibodies for 24 hours in the presence of HS-131. Compared to vehicle treated cells. HS-131 significantly reduced GM-CSF (p<0.001). IFNY (p<0.001). IL-17A (p<0.001). MIP-1a/B (p=0.013) and TNF a (p=0.025) (see. FIG. 3F).

### Example 2: In vivo Tracking of eHsp90 Expression in Autoimmune Disease States

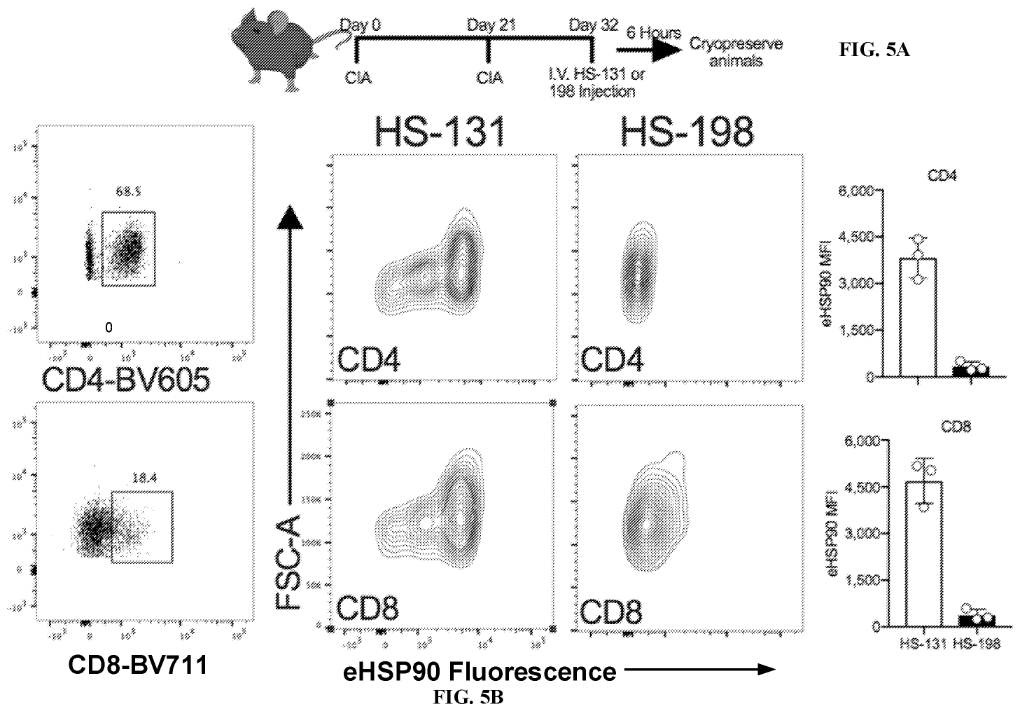

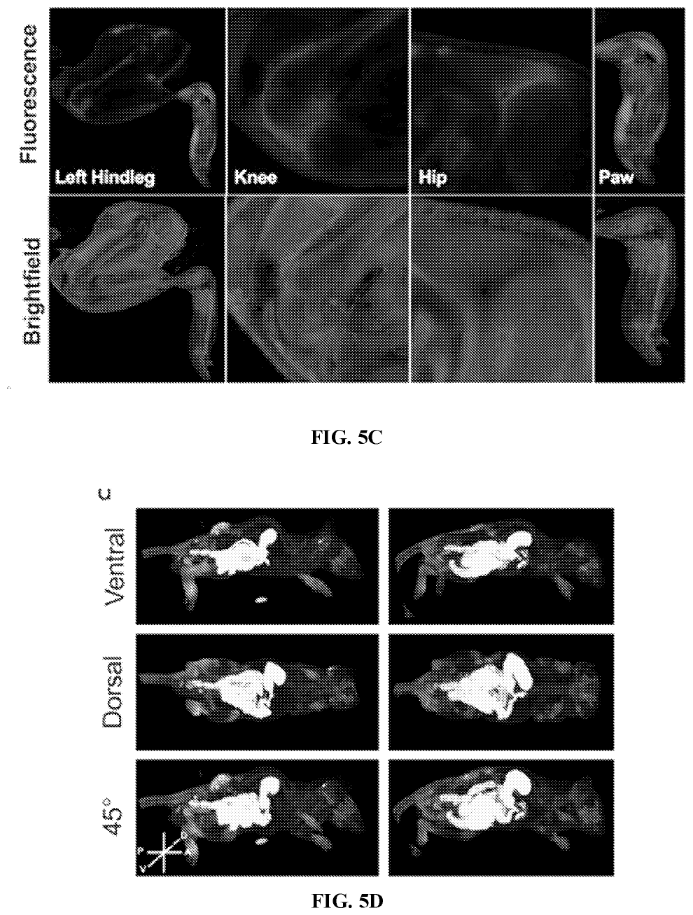

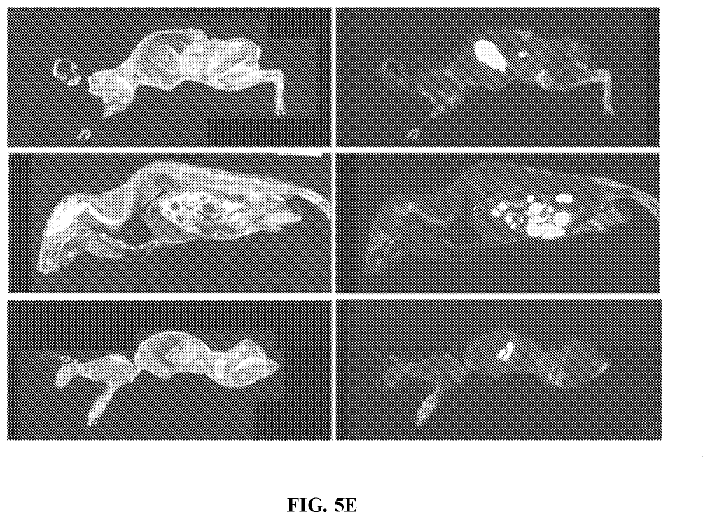

Previous work suggested that the expression of eHsp90 was restricted to tumor cells exhibiting a metastatic phenotype. This was consistently seen in cell culture, tumor cell xenografts and mouse models of spontaneous metastatic disease (Barrott et al. Chem Biol 20. 1187-1197 (2013)). The studies with isolated human PBMCs now suggest that activated T cells also upregulate eHsp90 in response to pro-inflammatory activation. To determine if this phenomenon occurred within the context of a fully functional immune system, the collagen induced arthritic (CIA) mouse model of human rheumatoid arthritis (RA) was utilized (Rice et al. Arthritis Rheum 58. 3765-3775 (2008)). Mice (DBA/IlacJ ˜8 weeks old) reliably develop polyarthritis when immunized against bovine type II collagen (Scarneo et al. Arthritis Res Ther 21, 292 (2019): Campbell et al., Ann Rheum Dis 56, 364-368 (1997)). The disease that occurs is usually non-symmetric, and any combination of paws/joints may be affected. Mice were injected via the tail vein with bovine collagen at Day (and then again 21 days later (FIG. 5A). This second injection triggered the development of the CIA related symptoms over the next several days within the joints. A primary mediator of local inflammatory responses in autoimmunity are invading T cell populations recognizing foreign antigens. To determine if invading T cells at sites of active disease expressed eHsp90, the animals were injected with HS-131 or HS-198. This was done at the peak of RA symptoms following the second immune challenge. After 6 hours, the animals were euthanized, immediately cryo-frozen in liquid nitrogen and cryo-sectioned (see, FIG. 5A).

Approximately, 500 40 μm slices were made and each slice imaged with both bright field and by fluorescence at Ex640 nm/Em680 nm. Evaluation of the biodistribution was then made by histological examination of individual slices and after in silico reconstruction of the entire mouse anatomy (see, FIGS. 5B-5E).

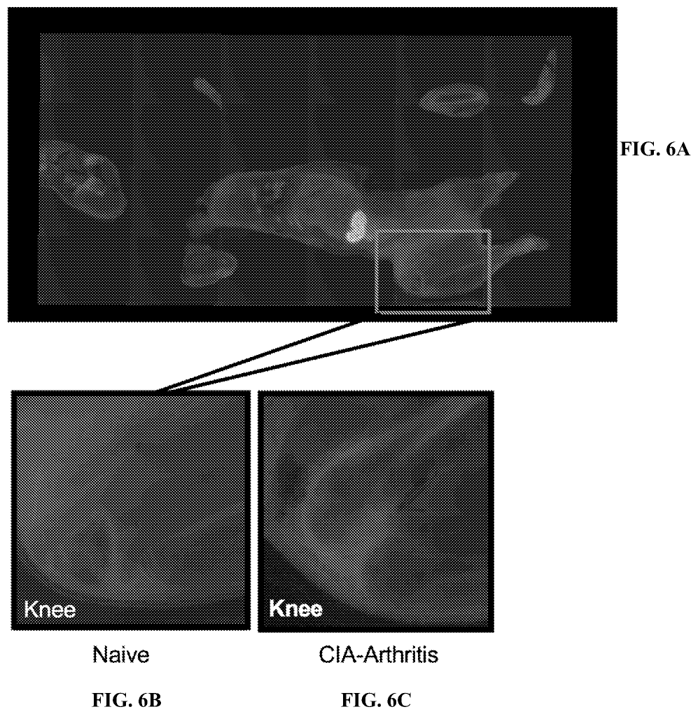

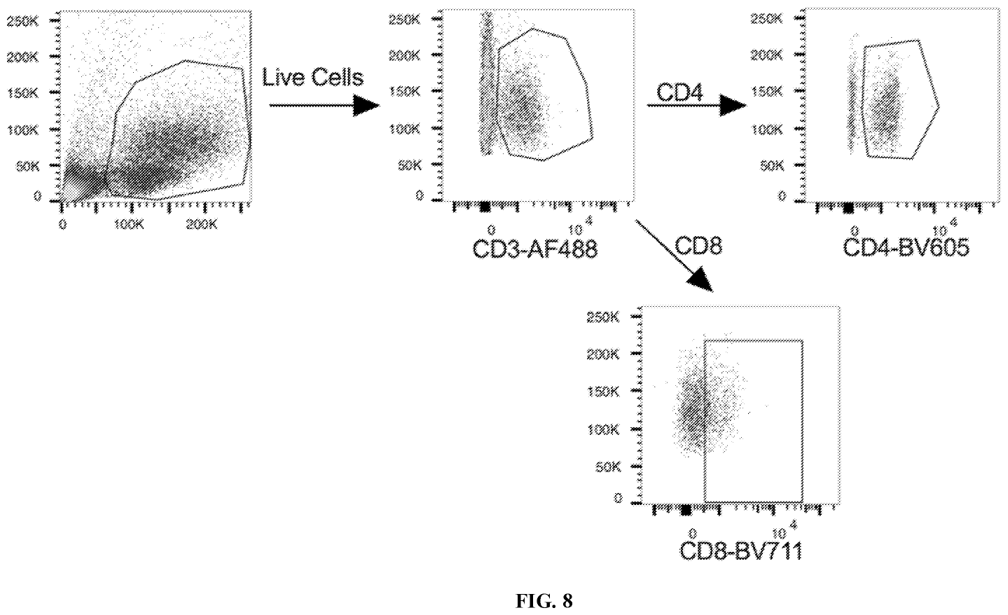

Immune cell phenotyping by flow cytometry of PBMC populations from the CIA mice showed pronounced eHsp90 expression across T-cells as measured by uptake of HS-131. For example, lymphocyte populations of inflammatory CD4 and CD8 cells both showed null, medium and high eHsp90 expression indicative of varying activation states in vivo (FIG. 5B and FIG. 8). No signal was obtained after flow studies on PBMCs isolated from CIA mice treated with HS-198, further showing the specificity of HS-131 for eHsp90 in vivo (FIG. 5B insert). Histological examination of joint slices taken from the cryo-sectioned CIA HS-131 mouse showed enhanced fluorescence in the joints of the animals, which also correlated with mean arthritic edema scoring and anatomic changes associated with local edema. In particular, the hind leg, knee, hip and paw all showed a marked increase in HS-131 localization, congruent with infiltration of significant lymphocyte and leukocyte populations (FIG. 5C). Analysis of the whole-body reconstruction of CIA mice shows that systemic HS-131 distribution was largely confined to areas of active immune cell invasion such as the hind paws and joints of the animals. Comparison of HS-131 uptake in a naïve non diseased animal shows no marked HS-131 in the joints including knee when compared to a CIA diseased animal (FIGS. 6A-6C). The sections shown in these figures also highlight the major route of HS-131 elimination, which is via the hepatobiliary system and intestines, similar to prior work in mice bearing breast tumors. Additionally, the fluorescence associated with the eye was also noted in the previous study. This is a natural fluorescence at Ex640 nm/Em680 nm associated with the Harderian glands found in rodents (Strum & Shear, Tissue Cell 14, 135-148 (1982)). Importantly, no fluorescence was observed in the joints or paws of the tumor-bearing animals in the prior studies.

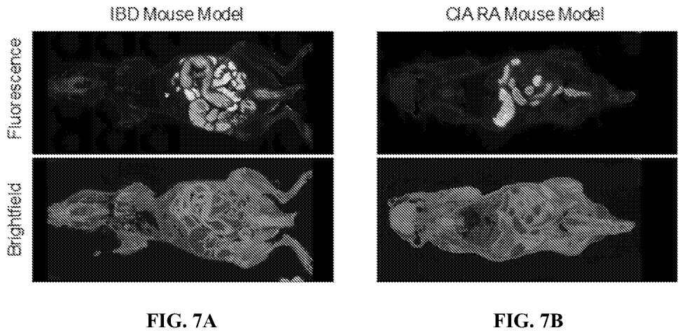

To further illustrate the specificity of HS-131 for sites of active immune cell localization, the cryo-sectioning studies were repeated in a mouse model of inflammatory bowel disease (IBD). In this model naïve T cells are isolated and transferred to SCID mice via intraperitoneal injection. Following T cell adoption, the mice developed symptoms of IBD similar to human disease including epithelial hyperplasia, extensive immune cell infiltration and distended colon (Read & Powrie, Curr Protoc Immunol, 15. 1-15 (2001)). Since HS-131 is exclusively eliminated through the biliary system and intestine, mice were imaged 24 hours post HS-131 tail vein injection in an effort to reduce the non-specific intestinal fluorescence signal associated with hepatobiliary probe clearance. Brightfield imaging of IBD mice showed extensive intestinal distention and inflammation consistent with the development of disease. Similarly, extensive fluorescent signaling from the inflamed bowels indicated areas of high eHsp90 expression consistent with infiltrating immune cell populations (FIG. 7A). In comparison, CIA mice 24 hours post HS-131 injection showed no discrete fluorescence associated with the lower intestine (FIG. 7B). Comparison of the biodistribution of HS-131 between the two auto-immune models also illustrated the specificity of the probe for sites of immune induced inflammation. In the case of the IBD mouse, no uptake of HS-131 was observed in any of the animal's joints or paws (FIGS. 7A-7B).

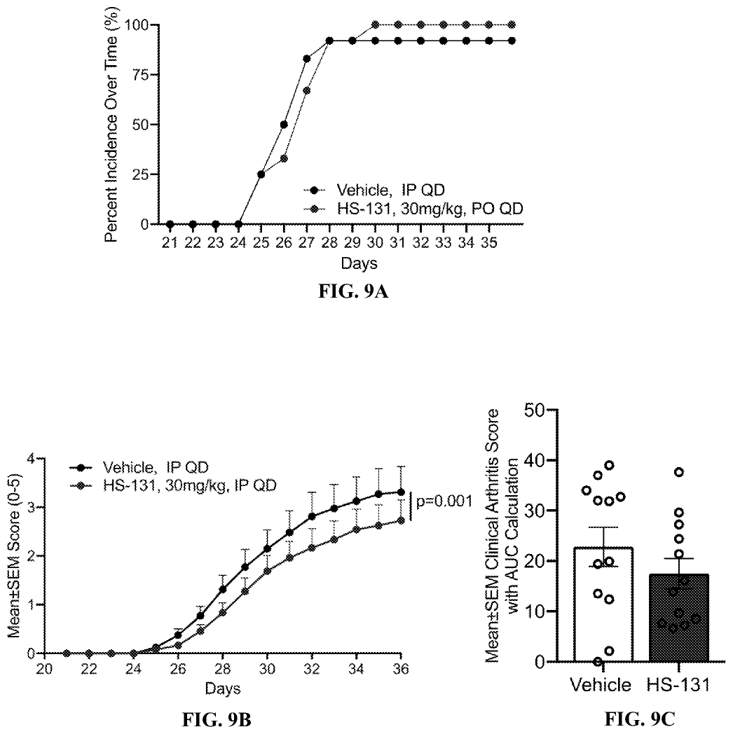

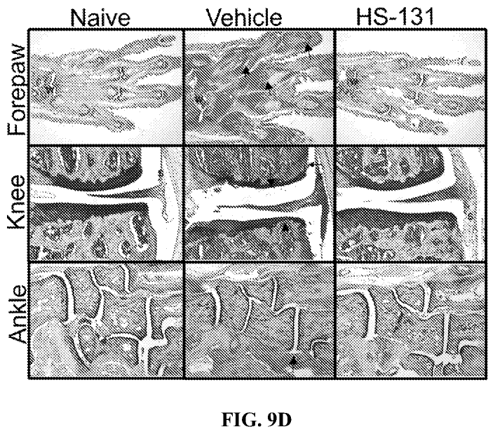

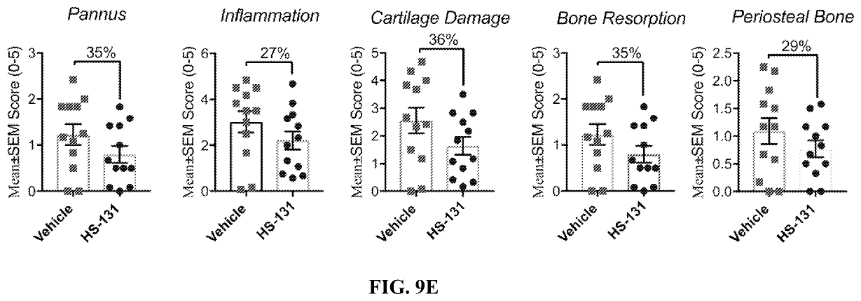

Previous studies have shown that non-tethered Hsp90 inhibitors may act as anti-inflammatory agents by suppressing pro-inflammatory cytokine production in stimulated immune cells (Rice et al., Arthritis Rheum 58, 3765-3775 (2008)). When administered to animals either orally or by injection, these small diffusible molecules are absorbed in all cell types systemically. In contrast, as shown herein and in prior work with tumor disease models, fluor-tethered inhibitors preferentially accumulate in cells expressing eHsp90. Therefore, to test the therapeutic potential of selectively targeting only eHsp90 as an immune-suppressant, HS-131 was tested for therapeutic efficacy in the CIA mouse model. First, the in vivo bioavailability and pharmacokinetics of HS-131 following i.p. administration was established. Upon daily dosing and following administration of the second collagen challenge, it was observed that the pathogenesis of disease development in the HS-131 treated group was comparable to the vehicle control (FIG. 9A). However, overall, treatment with HS-131 significantly reduced the mean clinical arthritic score of mice throughout the study period when compared to vehicle control (p<. 001) (FIG. 9B). Analysis of the area under the curve showed a ˜23% decrease in clinical score between the groups (FIG. 9C). Given the rapid clearance of fluor-tethered versions of Hsp90 inhibitors via the hepato-biliary system, it is remarkable that any efficacy was observed in the CIA model. Histopathologic effects of HS-131 were evaluated in the joints (knee, forepaw; ankle) of disease animals at the study terminus. Disease control animals had histopathologic changes consistent with those seen in type II collagen induced arthritis with microscopic alterations of synovium and periarticular tissue with neutrophils and mononuclear inflammatory cells (inflammation), marginal zone pannus and bone resorption and cartilage damage (chondrocyte death, proteoglycan loss and collagen matrix damage) (FIG. 9D). Histological scoring of the major joints effected by the CIA model showed HS-131 reduced the pannus (35%), inflammation (27%), cartilage damage (36%), bone resorption (35%) and periosteal bone (29%) compared to vehicle treated animals (FIG. 9E). These findings suggest that development of longer acting tethered inhibitors of Hsp90 is warranted to achieve a better therapeutic outcome. The promise of such drugs is that they would preferentially target activated immune cells expressing eHsp90 with potentially minimal side effects, in contrast to current broadly acting RA therapies such as TNF-α inhibitors (Feldmann, Nat Rev Immunol 2, 364-371 (2002): Monaco et al., Int Immunol 27, 55-62 (2015): Atzeni et al., Immunotherapy 7, 353-361 (2015)).

Discussion: T cells mediate many maladaptive disease pathologies such as autoimmune disorders. Many immunosuppressants in clinical use act by preventing the activation of T cells. However, this approach can lead to increased rates of opportunistic infection and malignancy. In the present disclosure, it was shown for the first time that eHsp90 was upregulated during T cell activation in response to exogenous ligands. Therefore, pharmacological inhibition of eHsp90 may provide a means to target only activated T cells for immunosuppression. Selective targeting of only activated T cells could be efficacious against autoimmune disease without increasing the incidence of undesirable outcomes. Additionally, the presence of eHsp90 on the surface of activated T cells could be diagnostically useful to track the status and progression of autoimmune disease in patients.

Heat shock protein 90 (Hsp90) maintains cellular proteostasis during stress and has been under investigation as a therapeutic target in cancer for over two decades. A membrane expressed or ectopic form of Hsp90 (eHsp90) was identified that previously appeared to be restricted to rapidly proliferating cells exhibiting a metastatic phenotype. In the present disclosure, HS-131, a fluor-tethered eHsp90 inhibitor, was used to quantify the effect of T cell activation on the expression of eHsp90 in human and mouse T cells. In cell-based assays, stimulation of human T cells induced a 20-fold increase in eHsp90 expression at the plasma membrane, suggesting trafficking of eHsp90 is acutely regulated by TCR and inflammatory mediated signaling. Following injection of HS-131 in mouse models of human rheumatoid arthritis (RA) and inflammatory bowel disease (IBD), localization of the probe at sites of active disease was detected, consistent with immune cell invasion. Moreover, despite rapid hepatobiliary clearance, HS-131 demonstrated efficacy in delaying the onset and progression of disease in the arthritis model. The results suggest eHsp90 expression at the plasma membrane of T cells is a molecular marker of autoimmune induced activation and potentially a therapeutic target for chronic diseases such as rheumatoid arthritis and inflammatory bowel disease.

The present disclosure shows that eHsp90 expression was dramatically upregulated in activated T cell populations in response to cytokine challenge or in vivo autoimmune activation. Thus, with respect to expression of eHsp90, activated T cell populations behave similarly to tumor cells exhibiting a malignant phenotype (Tsutsumi & Neckers, Cancer Sci 98, 1536-1539 (2007): Crowe et al., ACS Chem Biol 12, 1047-1055 (2017)). By contrast, even in a highly stressed systemic disease state, such as mouse models of RA and IBD, quiescent or fully differentiated cells do not express eHsp90 or absorb HS-131. In malignancy, the precise molecular mechanism governing expression of eHsp90 has yet to be defined. However, it has been shown that over-expression of known oncogenic protein kinases such as HER2 can induce benign tumor cells to express Hsp90 at the plasma membrane (Crowe et al., ACS Chem Biol 12, 1047-1055 (2017): Citri et al., EMBO Rep 5, 1165-1170 (2004): Solit et al., Clin Cancer Res 8, 986-993 (2002)). The present studies with IL-2 demonstrated for the first time that eHsp90 trafficking can be actively regulated through receptor mediated signaling. This process is not merely the consequence of overexpressed Hsp90 due to a general cellular stress response. A possible role for eHsp90 in T cells is the chaperoning of CD surface proteins like CD3, CD4 and CD8, since it has been noted that loss of trafficking of these proteins to the cell surface in the presence of diffusible Hsp90 inhibitors (Bae et al., J Immuno/190, 1360-1371 (2013)). Additionally, the in vivo work in animal models of autoimmune disease has shown that HS-131 is able to label sites of edema and inflammation, which potentially provides a new way to identify and monitor active inflammatory disease.

The potential of HS-131 to reduce disease activity in autoimmune diseases was further tested. Despite rapid biliary clearance, a small but statistically significant reduction and delay of symptoms was observed in the CIA mouse model of human RA. These findings suggest that longer acting eHsp90 targeted inhibitors could be effective and precise antiarthritic drugs. In this context, targeting eHsp90) may selectively remove auto-antigenic T cell populations permanently, perhaps even those involved in T cell memory. The studies with fluor-tethered inhibitors of Hsp90, first in cancer and now immune disease models, has shown that expression of eHsp90 is associated with rapidly proliferating cells and not fully differentiated cell populations or those preprogramed to be actively replaced such as gut epithelium. T cells are essential to immune system's ability to fight infections: however, many diseases confer maladaptive T cell activation including RA, lupus and IBS as well as other autoimmune diseases. If the hypothesis is correct, the unique association of eHsp90 expression with T cell activation opens up several avenues that have both diagnostic and therapeutic relevance with respect to autoimmune disease, managing transplant rejection and certain viral infections. For diagnostic purposes, with the advent of fluor-tethered Hsp90 inhibitors, one can now track T cell activation in blood samples in response to infection or in autoimmune disease. Ultimately these results suggest that further evaluation of the therapeutic potential of Hsp90 inhibitors in autoimmune disease is warranted. If one could intervene at the time of activation in certain autoimmune diseases that are associated with infection, such as type I diabetes or chronic Lyme disease, perhaps one could prevent the aberrant T cell “education” that ultimately gives rise to underlying chronic pathology associated with these infections. In situations of active ongoing chronic disease, tethered eHsp90 inhibitors carrying toxins or radionucleotides could be employed to acutely, precisely eliminate host targeting T cell populations without affecting the quiescent population. Once the inhibitor had been cleared by the hepatobiliary system, the patient would not be expected to suffer from long-term immunosuppression.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

What is claimed is:

1. A method of preventing or treating an autoimmune disease in a subject, the method comprising administering to the subject a compound capable of inhibiting eHsp90.

2. The method of claim 1, wherein the compound has the following general formula (I): 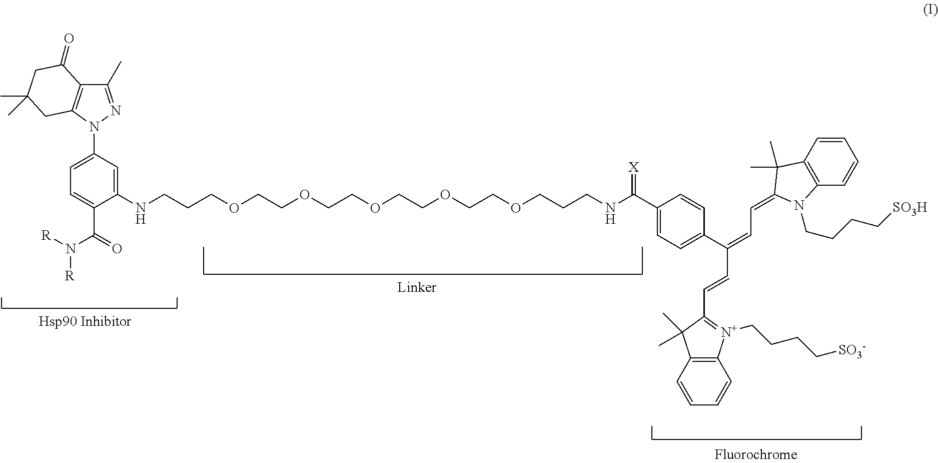
wherein R is H or C₁₋₈-alkyl; and X is H₂, O, or S.

3. The method of claim 2, wherein R is H, and X is H₂.

4. The method of claim 2, wherein R is H, and X is O.

5. The method of claim 1, wherein the autoimmune disease is a disease that confers maladaptive T cell activation.

6. The method of claim 5, wherein the autoimmune disease is selected from the group consisting of rheumatoid arthritis, osteoarthritis, systemic scleroderma, Chron's disease, systemic lupus erythematosus, inflammatory bowel disease, Type I diabetes, chronic Lyme disease, Addison disease, celiac disease, dermatomyositis, Graves disease, Hashimoto thyroiditis, multiple sclerosis, Myasthenia gravis, Pernicious anemia, reactive arthritis, and Sjogren syndrome.

7. The method of any one of claims 1-6, further comprising administering to the subject one or more additional autoimmune therapies.

8. A method of diagnosing an autoimmune disease in a subject, the method comprising:
(a) obtaining from the subject a biological sample; and
(b) contacting the biological sample with a compound capable of inhibiting eHsp90,
wherein detection of elevated T cell activation in the biological sample is indicative of presence of an autoimmune disease in the subject.

9. The method of claim 8, wherein the compound has the following general formula (I): 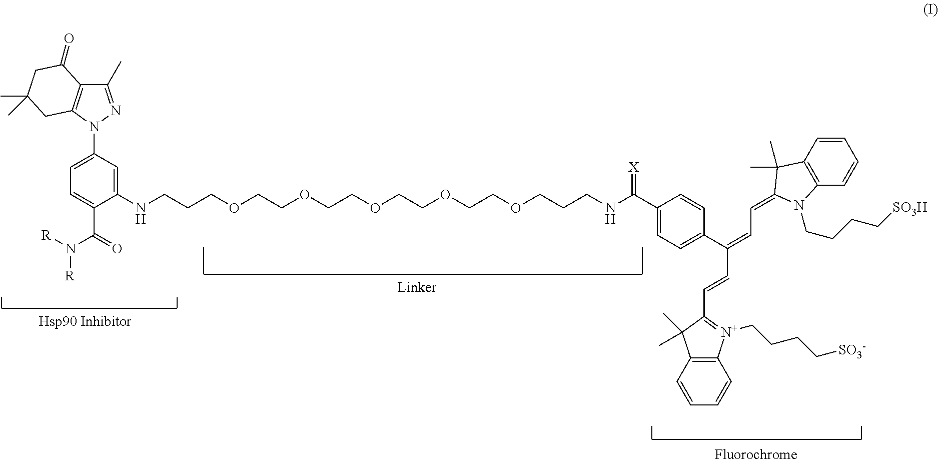
wherein R is H or C₁₋₃-alkyl; and X is H₂, O, or S.

10. The method of claim 9, wherein R is H, and X is H₂.

11. The method of claim 9, wherein R is H, and X is O.

12. The method of claim 8, wherein the autoimmune disease is a disease that confers maladaptive T cell activation.

13. The method of claim 12, wherein the autoimmune disease is selected from the group consisting of rheumatoid arthritis, osteoarthritis, systemic scleroderma, Chron's disease, systemic lupus erythematosus, inflammatory bowel disease, Type I diabetes, chronic Lyme disease, Addison disease, celiac disease, dermatomyositis, Graves disease, Hashimoto thyroiditis, multiple sclerosis, Myasthenia gravis, Pernicious anemia, reactive arthritis, and Sjogren syndrome.

14. A method of delaying onset and progression of an autoimmune disease in a subject, the method comprising administering to the subject a compound capable of inhibiting eHsp90.

15. The method of claim 14, wherein the compound has the following general formula (I): 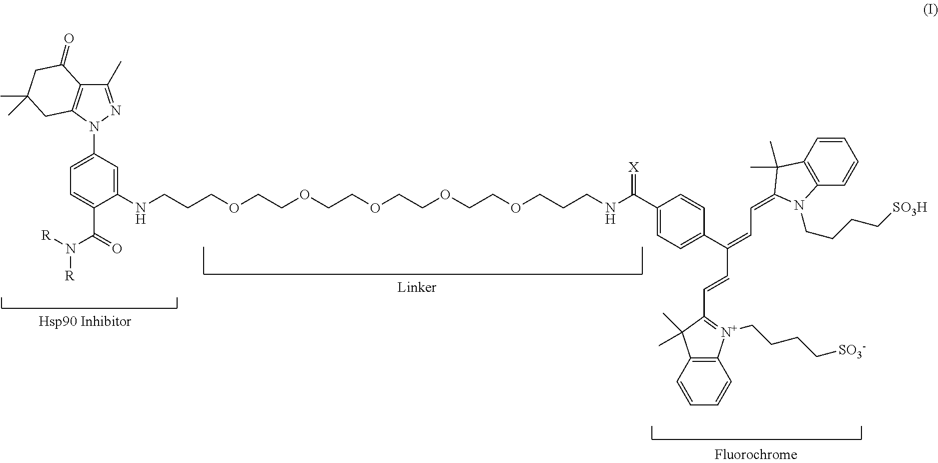
wherein R is H or C₁₋₈-alkyl; and X is H₂, O, or S.

16. The method of claim 15, wherein R is H, and X is H₂.

17. The method of claim 15, wherein R is H, and X is O.

18. The method of claim 14, wherein the autoimmune disease is a disease that confers maladaptive T cell activation.

19. The method of claim 18, wherein the autoimmune disease is selected from the group consisting of rheumatoid arthritis, osteoarthritis, systemic scleroderma, Chron's disease, systemic lupus erythematosus, inflammatory bowel disease, Type I diabetes, chronic Lyme disease, Addison disease, celiac disease, dermatomyositis, Graves disease, Hashimoto thyroiditis, multiple sclerosis, Myasthenia gravis, Pernicious anemia, reactive arthritis, and Sjogren syndrome.

20. The method of any one of claims 14-19, further comprising administering to the subject one or more additional autoimmune therapies.

21. A method of managing a transplant rejection in a subject, the method comprising administering to the subject a compound capable of inhibiting eHsp90 thereby tracking T cell activation.

22. The method of claim 21, wherein the compound has the following general formula (I): 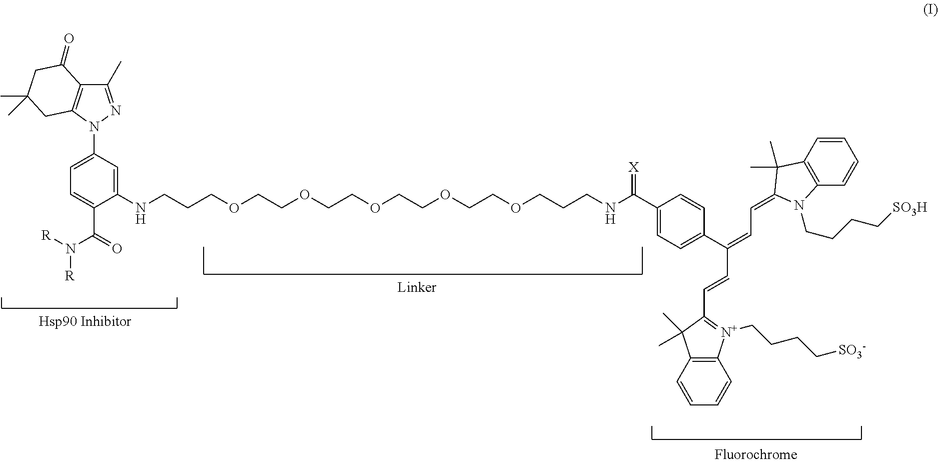
wherein R is H or C₁₋₈-alkyl; and X is H₂, O, or S.

23. The method of claim 22, wherein R is H, and X is H₂.

24. The method of claim 22, wherein R is H, and X is O.

25. A method of managing an infection in a subject, the method comprising administering to the subject a compound capable of inhibiting eHsp90 thereby tracking T cell activation.

26. The method of claim 25, wherein the compound has the following general formula (I): 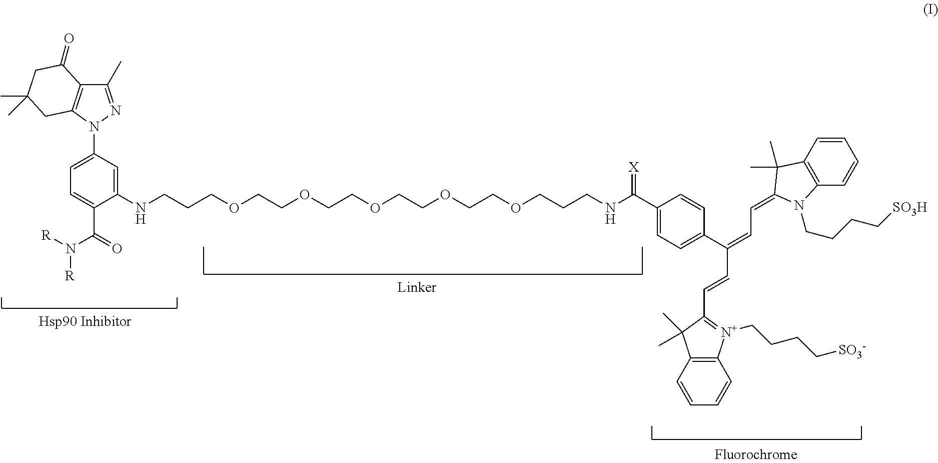
wherein R is H or C₁₋₈-alkyl; and X is H₂, O, or S.

27. The method of claim 26, wherein R is H, and X is H₂.

28. The method of claim 26, wherein R is H, and X is O.
